(19) United States
(12) Patent Application Publication
(10) Pub. No.: US 2004/0157225 A1
(43) Pub. Date: Aug. 12, 2004

(54) Diagnostic and therapeutic use of a nuclear restricted protein for alzheimer's disease and related neurodegenerative disorders

(76) Inventors: Rainer Hipfel, Heidelberg (DE); Heinz Von Der Kammer, Hamburg (DE); Johannes Pohlner, Hamburg (DE)

Correspondence Address: JACOBSON HOLMAN PLLC, 400 SEVENTH STREET N.W., SUITE 600, WASHINGTON, DC, 20004, US

(21) Appl. No.: 10/474,035

(22) PCT Filed: Apr. 13, 2002
(86) PCT No.: PCT/EP02/04136

(30) Foreign Application Priority Data
Apr. 14, 2001 (EP) 01109230.1

(51) Int. Cl. C12Q 1/68

(52) U.S. Cl. 435/6

(57) ABSTRACT

The present invention discloses the differential expression of the gene coding for the nuclear restricted protein/brain, NRP/B, in specific brain regions of Alzheimer's disease patients. Based on this finding this invention provides a method for diagnosing or prognosticating a neurodegenerative disease, in particular Alzheimer's disease, in a subject, or for determining whether a subject is at increased risk of developing such a disease. Furthermore, this invention provides therapeutic and prophylactic methods for treating or preventing Alzheimer's disease and related neurodegenerative disorders using a gene coding for a nuclear restricted protein, in particular the nuclear restricted protein/brain, NRP/B. A method of screening for modulating agents of neurodegenerative diseases is also disclosed.

The present invention relates to methods of diagnosing, prognosticating and monitoring the progression of neurodegenerative diseases in a subject. Furthermore, methods of therapy control and screening for modulating agents of neurodegenerative diseases are provided. The invention also discloses pharmaceutical compositions, kits and recombinant animal models.

Neurodegenerative diseases, in particular Alzheimer's disease (AD), have a strongly debilitating impact on a patient's life. Furthermore, these diseases constitute an enormous health, social and economic burden. AD is the most common age-related neurodegenerative condition affecting about 10% of the population over 65 years of age and up to 45% over age 85 (for a recent review see Vickers et al., Progress in Neurobiology 2000, 60:139-165). Presently, this amounts to an estimated 12 million cases in the US, Europe, and Japan. This situation will inevitably worsen with the demographic increase in the number of old people (“aging of the baby boomers”) in developed countries. The neuropathological hallmarks that occur in the brains of individuals with AD are senile plaques, composed of amyloid-b protein, and profound cytoskeletal changes coinciding with the appearance of abnormal filamentous structures and the formation of neurofibrillary tangles. AD is a progressive disease that is associated with early deficits in memory formation and ultimately leads to the complete erosion of higher cognitive function. A characteristic feature of the pathogenesis of AD is the selective vulnerability of particular brain regions and subpopulations of nerve cells to the degenerative process. Specifically, the temporal lobe region and the hippocampus are affected early and more severely during the progression of the disease. On the other hand, neurons within the frontal cortex, occipital cortex, and the cerebellum remain largely intact and are protected from neurodegeneration (Terry et al., Annals of Neurology 1981, 10:184-192).

Currently, there is no cure for AD, nor is there an effective treatment to halt the progression of AD or even a method to diagnose AD antemortem with high probability. Several risk factors have been identified that predispose an individual to develop AD, among them most prominently the epsilon4 allele of apolipoprotein E (ApoE). Although there are rare examples of early-onset AD which have been attributed to genetic defects in the genes for the amyloid precursor protein (APP), presenilin-1, and presenilin-2, the prevalent form of late-onset sporadic AD is of hitherto unknown etiologic origin. The late onset and complex pathogenesis of neurodegenerative disorders pose a formidable challenge to the development of therapeutic and diagnostic agents. It is crucial to expand the pool of potential drug targets and diagnostic markers. It is therefore an object of the present invention to provide methods, materials, and animal models which are suited inter alia for the diagnosis and development of a treatment of AD or related neurodegenerative diseases. This object has been solved by the features of the independent claims. The subclaims define preferred embodiments of the present invention.

The gene coding for the nuclear restricted protein/brain (NRP/B) was originally cloned by Kim et al. (Journal of Cell Biology 1998, 141:553-566) in an attempt to identify proteins that may play a role in brain development and neuronal differentiation. The human NRP/B gene (Genbank Accession No. NM 003633) codes for a 589-amino acid protein with a predicted molecular weight of 67 kDa. NRP/B shares no homology or similarity to any other known proteins, and its amino acid sequence is highly conserved between human and mouse. At its N-terminus the NRP/B protein possesses a BTB domain-like structure (˜35% identity) which has been implicated in protein-protein interactions involving the cytoskeleton. The C-terminus shows some homology (˜28% identity) to a “kelch motif” which is shared among several actin-associated proteins. The NRP/B gene is expressed as a 5.5 kb mRNA predominantly in human fetal and adult brain with modest expression in a few other fetal tissues such as heart, kidney, and lung, and very low levels of expression in adult pancreas. In the human adult brain, expression is particularly prominent in the hippocampus, amygdala, and cerebral cortex. The NRP/B protein can exist in two forms. A 67 kDa form and a 57 kDa form are detected in total cell lysates, whereas in the nuclear fraction only the 67 kDa form is observed. In the nucleus NRP/B seems to be associated with the nuclear matrix.

A functional analysis of NRP/B by Kim et al. (ref. see above) suggests a participation of NRP/B in the regulation of neuronal differentiation and process formation. This notion is based on the finding that NRP/B expression is up-regulated during neuronal differentiation, and that over-expression of NRP/B augments neuronal process formation in cell culture experiments. Furthermore, NRP/B antisense inhibition experiments in rat primary hippocampal neurons impedes neurite development. Another functional aspect of NRP/B is its physical association with the functionally active, hypophosphorylated form of the p110^{RB} retinoblastoma protein during neuronal differentiation of human SH-SY5Y neuroblastoma cells induced by retinoic acid. The hypophosphorylated form of p110^{RB} is also found to be associated with the nuclear matrix, and over-expression of p110^{RB} can induce neuronal differentiation. Thus, NRP/B could play a potential role in the regulation of the cell cycle by interfering with other cell cycle regulatory proteins such as p110^{RB}. This invention is based on the differential expression of the gene coding for NRP/B in brain samples of AD patients. More specifically, the present invention discloses a differential up-regulation of NRP/B gene expression in the frontal lobe region of AD patients relative to samples derived from the temporal cortex region. No such up-regulation is observed in samples from age-matched healthy controls. To date, no experiments have been described that show a relationship between a differential expression of the gene coding for NRP/B and the pathology of neurodegenerative diseases, particularly AD. Such a link offers new ways, inter alia, for the diagnosis and treatment of said disorders.

The singular forms “a”, “an”, and “the” as used herein and in the claims include plural reference unless the context dictates otherwise. For example, “a cell” means as well a plurality of cells, and so forth. The term “and/or” as used in the present specification and in the claims implies that the phrases before and after this term are to be considered either as alternatives or in combination. For instance, the wording “determination of a level and/or an activity” means that either only a level, or only an activity, or both a level and an activity are determined. The term “level” as used herein is meant to comprise a gage of, or a measure of the amount of, or a concentration of a transcription product, for instance an mRNA, or a translation product, for instance a protein or polypeptide. The term “activity” as used herein shall be understood as a measure for the ability of a transcription product or a translation product to produce a biological effect or a measure for a level of biologically active molecules. The term “activity” also refers to enzymatic activity. The terms “level” and/or “activity” as used herein further refer to gene expression levels or gene activity. Gene expression can be defined as the utilization of the information contained in a gene by transcription and translation leading to the production of a gene product. A gene product comprises either RNA or protein and is the result of expression of a gene. The amount of a gene product can be used to measure how active a gene is. The term “gene” as used in the present specification and in the claims comprises both coding regions (exons) as well as non-coding regions (e.g. non-coding regulatory elements such as promoters or enhancers, introns, leader and trailer sequences). The term “fragment” as used herein is meant to comprise e.g. an alternatively spliced, or truncated, or otherwise cleaved transcription product or translation product. The term “derivative” as used herein refers to a mutant, or an RNA-edited, or a chemically modified, or otherwise altered transcription product, or to a mutant, or chemically modified, or otherwise altered translation product. For instance, a “derivative” may be generated by processes such as altered phosphorylation, or glycosylation, or lipidation, or by altered signal peptide cleavage or other types of maturation cleavage. These processes may occur post-translationally. The term “modulator” as used in the present invention and in the claims refers to a molecule capable of changing or altering the level and/or the activity of a gene, or a transcription product of a gene, or a translation product of a gene. Preferably, a “modulator” is capable of changing or altering the biological activity of a transcription product or a translation product of a gene. Said modulation, for instance, may be an increase or a decrease in enzyme activity, a change in binding characteristics, or any other change or alteration in the biological, functional, or immunological properties of said translation product of a gene. The term ‘AD’ shall mean Alzheimer's disease.

Neurodegenerative diseases or disorders according to the present invention comprise Alzheimer's disease, Parkinson's disease, Huntington's disease, amyotrophic lateral sclerosis, Pick's disease, fronto-temporal dementia, progressive nuclear palsy, corticobasal degeneration, cerebro-vascular dementia, multiple system atrophy, and mild-cognitive impairment. Further conditions involving neurodegenerative processes are, for instance, ischemic stroke, age-related macular degeneration and narcolepsy.

In one aspect, the invention features a method of diagnosing or prognosticating a neurodegenerative disease in a subject, or determining whether a subject is at increased risk of developing said disease. The method comprises: determining a level, or an activity, or both said level and said activity of (i) a transcription product of a gene coding for a nuclear restricted protein, and/or of (ii) a translation product of a gene coding for a nuclear restricted protein, and/or of (iii) a fragment or derivative of said transcription or translation product in a sample from said subject and comparing said level, and/or said activity to a reference value representing a known disease or health status, thereby diagnosing or prognosticating said neurodegenerative disease in said subject, or determining whether said subject is at increased risk of developing said neurodegenerative disease.

In a further aspect, the invention features a method of monitoring the progression of a neurodegenerative disease in a subject. A level, or an activity, or both said level and said activity, of (i) a transcription product of a gene coding for a nuclear restricted protein, and/or of (ii) a translation product of a gene coding for a nuclear restricted protein, and/or of (iii) a fragment or derivative of said transcription or translation product in a sample from said subject is determined. Said level and/or said activity is compared to a reference value representing a known disease or health status. Thereby the progression of said neurodegenerative disease in said subject is monitored.

In still a further aspect, the invention features a method of evaluating a treatment for a neurodegenerative disease, comprising determining a level, or an activity, or both said level and said activity of (i) a transcription product of a gene coding for a nuclear restricted protein, and/or of (ii) a translation product of a gene coding for a nuclear restricted protein, and/or of (iii) a fragment or derivative of said transcription or translation product in a sample obtained from a subject being treated for said disease. Said level, or said activity, or both said level and said activity are compared to a reference value representing a known disease or health status, thereby evaluating the treatment for said neurodegenerative disease. In a preferred embodiment, said subjects suffer from Alzheimer's disease.

It is preferred that said nuclear restricted protein is NRP/B (nuclear restricted protein/brain). The present invention discloses the differential expression and regulation of the gene coding for the nuclear restricted protein/brain in specific brain regions of AD patients. Consequently, the NRP/B gene and its corresponding translation products may have a causative role in the regional selective neuronal degeneration typically observed in AD. Alternatively, NRP/B may confer a neuroprotective function to the remaining surviving nerve cells. Based on these disclosures, the present invention has utility for the diagnostic evaluation and prognosis as well as for the identification of a predisposition to a neurodegenerative disease, in particular AD. Furthermore, the present invention is useful for the diagnostic monitoring of patients undergoing treatment for such a disease.

It is a preferred embodiment that said sample of a subject to be analyzed and determined is selected from the group consisting of a brain tissue or other tisssue, organs, or body cells. The sample can preferably consist of cerebrospinal fluid or other body fluids such as saliva, urine, blood, serum plasma, or nasal mucosa.

In further preferred embodiments, said reference value is that of a level, or an activity, or both said level and said activity of (i) a transcription product of a gene coding for a nuclear restricted protein, and/or of (ii) a translation product of a gene coding for a nuclear restricted protein, and/or of (iii) a fragment or derivative of said transcription or translation product in a sample from a subject not suffering from said neurodegenerative disease.

In preferred embodiments, an increase or decrease of a transcription product of a gene coding for NRP/B and/or a translation product of a gene coding for NRP/B in a sample cell or tissue from said subject relative to a reference value representing a known health status indicates a diagnosis, or prognosis, or increased risk of becoming diseased with a neurodegenerative disease, particularly Alzheimer's disease.

In preferred embodiments, measurement of a level of transcription products of a gene coding for a nuclear restricted protein is performed in a sample from a subject using a quantitative PCR-analysis with primer combinations to amplify said gene specific sequences from cDNA obtained by reverse transcription of RNA extracted from a sample of a subject. A Northern blot with probes specific for said gene can also be applied. It might further be preferred to measure transcription products by means of chip-based micro-array technologies. These techniques are known to those of ordinary skill in the art (see Sambrook and Russell, Molecular Cloning: A Laboratory Manual, Cold Spring Harbor Laboratory Press, Cold Spring Harbor, N.Y., 2000).

Furthermore, a level and/or activity of a translation product of a gene coding for a nuclear restricted protein and/or fragment of said translation product can be detected using an immunoassay, an activity assay, and/or binding assay. These assays can measure the amount of binding between said protein molecule and an anti-protein antibody by the use of enzymatic, chromodynamic, radioactive, magnetic, or luminescent labels which are attached to either the anti-protein antibody or a secondary antibody which binds the anti-protein antibody. In addition, other high affinity ligands may be used. Immunoassays which can be used include e.g. ELISAs, Western blots and other techniques known to those of ordinary skill in the art (see Harlow and Lane, Antibodies: A Laboratory Manual, Cold Spring Harbor Laboratory Press, Cold Spring Harbor, N.Y., 1999). All these detection techniques may also be employed in the format of micro-arrays, protein-arrays, or protein-chip based technologies.

In a preferred embodiment the level, or the activity, or both said level and said activity of (i) a transcription product of a gene coding for a nuclear restricted protein, and/or of (ii) a translation product of a gene coding for a nuclear restricted protein, and/or of (iii) a fragment or derivative of said transcription or translation product in a series of samples taken from said subject over a period of time is compared, in order to monitor the progression of said disease. In further preferred embodiments, said subject receives a treatment prior to one or more of said sample gatherings. In yet another preferred embodiment, said level and/or activity is determined before and after said treatment of said subject.

In another aspect, the invention features a kit for diagnosing or prognosticating neurodegenerative diseases, in particular AD, in a subject, or determining the propensity or predisposition of a subject to develop a neurodegenerative disease, in particular AD, said kit comprising:

(a) at least one reagent which is selected from the group consisting of (i) reagents that selectively detect a transcription product of a gene coding for a nuclear restricted protein (ii) reagents that selectively detect a translation product of a gene coding for a nuclear restricted protein; and

(b) instruction for diagnosing, or prognosticating a neurodegenerative disease, in particular AD, or determining the propensity or predisposition of a subject to develop such a disease by

detecting a level, or an activity, or both said level and said activity, of said transcription product and/or said translation product of a gene coding for a nuclear restricted protein, in a sample from said subject; and

diagnosing or prognosticating a neurodegenerative disease, in particular AD, or determining the propensity or predisposition of said subject to develop such a disease,

wherein a varied level, or activity, or both said level and said activity, of said transcription product and/or said translation product compared to a reference value representing a known health status; or a level, or activity, or both said level and said activity, of said transcription product and/or said translation product similar or equal to a reference value representing a known disease status, indicates a diagnosis or prognosis of a neurodegenerative disease, in particular AD, or an increased propensity or predisposition of developing such a disease. The kit, according to the present invention, may be particularly useful for the identification of individuals that are at risk of developing a neurodegenerative disease, in particular AD. Consequently, the kit, according to the invention, may serve as a means for targeting identified individuals for early preventive measures or therapeutic intervention prior to disease onset, before irreversible damage in the course of the disease has been inflicted. Furthermore, in preferred embodiments, the kit featured in the invention is useful for monitoring a progression of a neurodegenerative disease, in particular AD, in a subject, as well as monitoring success or failure of therapeutic treatment for such a disease of said subject.

In another aspect, the invention features a method of treating or preventing a neurodegenerative disease, in particular AD, in a subject comprising the administration to said subject in a therapeutically or prophylactically effective amount of an agent or agents which directly or indirectly affect a level, or an activity, or both said level and said activity, of (i) a gene coding for a nuclear restricted protein, and/or (ii) a transcription product of a gene coding for a nuclear restricted protein, and/or (iii) a translation product of a gene coding for a nuclear restricted protein, and/or (iv) a fragment or derivative of (i) to (iii). Said agent may comprise a small molecule, or it may also comprise a peptide, an oligopeptide, or a polypeptide. Said peptide, oligopeptide, or polypeptide may comprise an amino acid sequence of a translation product of a gene coding for a nuclear restricted protein, or a fragment, or derivative, or a variant thereof.

In preferred embodiments, the method comprises the application of per se known methods of gene therapy and/or antisense nucleic acid technology to administer said agent or agents. In general, gene therapy includes several approaches: molecular replacement of a mutated gene, addition of a new gene resulting in the synthesis of a therapeutic protein, and modulation of endogenous cellular gene expression by recombinant expression methods or by drugs. Gene-transfer techniques are described in detail (see e.g. Behr, Acc Chem Res 1993, 26:274-278 and Mulligan, Science, 1993, 260: 926-93) and include direct gene-transfer techniques such as mechanical microinjection of DNA into a cell as well as indirect techniques employing biological vectors (like recombinant viruses, especially retroviruses) or model liposomes, or techniques based on transfection with DNA coprecipitation with polycations, cell membrane pertubation by chemical (solvents, detergents, polymers, enzymes) or physical means (mechanic, osmotic, thermic, electric shocks). The postnatal gene transfer into the central nervous system has been described in detail (see e.g. Wolff, Curr Opin. Neurobiol 1993, 3:743-748).

In particular, the invention features a method of treating or preventing a neurodegenerative disease by means of antisense nucleic acid therapy, i.e. the down-regulation of an inappropriately expressed or defective gene by the introduction of antisense nucleic acids or derivatives thereof into certain critical cells (see e.g. Gillespie, DN&P 1992, 5:389-395; Agrawal and Akhtar, Trends Biotechnol 1995, 13:197-199; Crooke, Biotechnology 1992, 10:882-6). Apart from hybridization strategies, the application of ribozymes, i.e. RNA molecules that act as enzymes, destroying RNA that carries the message of disease has also been described (see e.g. Barinaga, Science 1993, 262:1512-1514). In preferred embodiments, the subject to be treated is a human, and therapeutic antisense nucleic acids or derivatives thereof are directed against a human nuclear restricted protein, particularly NRP/B. It is preferred that cells of the central nervous system, preferably the brain, of a subject are treated in such a way. Cell penetration can be performed by known strategies such as coupling of antisense nucleic acids and derivatives thereof to carrier particles, or the above described techniques. Strategies for administering targeted therapeutic oligodeoxynucleotides are known to those of skill in the art (see e.g. Wickstrom, Trends Biotechnol, 1992, 10: 281-287). In some cases, delivery can be performed by mere topical application. Further approaches are directed to intracellular expression of antisense RNA. In this strategy, cells are transformed ex vivo with a recombinant gene that directs the synthesis of an RNA that is complementary to a region of target nucleic acid. Therapeutical use of intracellularly expressed antisense RNA is procedurally similar to gene therapy.

In further preferred embodiments, the method comprises grafting donor cells into the central nervous system, preferably the brain, of said subject, or donor cells preferably treated so as to minimize or reduce graft rejection, wherein said donor cells are genetically modified by insertion of at least one transgene encoding said agent or agents. Said transgene might be carried by a viral vector, in particular a retroviral vector. The transgene can be inserted into the donor cells by a nonviral physical transfection of DNA encoding a transgene, in particular by microinjection. Insertion of the transgene can also be performed by electroporation, chemically mediated transfection, in particular calcium phosphate transfection, liposomal mediated transfection, etc.

In preferred embodiments, said agent for treating and preventing a neurodegenerative disease, in particular AD, is a therapeutic protein which can be administered to said subject, preferably a human, by a process comprising introducing subject cells into said subject, said subject cells having been treated in vitro to insert a DNA segment encoding said therapeutic protein, said subject cells expressing in vivo in said subject a therapeutically effective amount of said therapeutic protein. Said DNA segment can be inserted into said cells in vitro by a viral vector, in particular a retroviral vector.

Methods of treatment, according to the present invention, comprise the application of therapeutic cloning, transplantation, and stem cell therapy using embryonic stem cells, or embryonic germ cells and neuronal adult stem cells, combined with any of the previously described cell and gene therapeutic methods. Stem cells may be totipotent or pluripotent. They may also be organ-specific. Strategies for repairing diseased and/or damaged brain cells or tissue comprise (i) taking donor cells from an adult tissue. Nuclei of those cells are transplanted into unfertilized egg cells from which the genetic material has been removed. Embryonic stem cells are isolated from the blastocyst stage of the cells which underwent somatic cell nuclear transfer. Use of differentiation factors then leads to a directed development of the stem cells to specialized cell types, preferably neuronal cells (Lanza et al., Nature Medicine 1999, 9: 975-977), or (ii) purifying adult stem cells, isolated from the central nervous system, or from bone marrow (mesenchymal stem cells), for in vitro expansion and subsequent grafting and transplantation, or (iii) directly inducing endogenous neural stem cells to proliferate, migrate, and differentiate into functional neurons (Peterson D A, Curr. Opin. Pharmacol. 2002, 2: 34-42). Adult neural stem cells are of great potential for repairing damaged or diseased brain tissues, as the germinal centers of the adult brain are free of neuronal damage or dysfunction (Colman A, Drug Discovery World 2001, 7: 66-71).

In preferred embodiments, the subject for treatment or prevention, according to the present invention, can be a human, an experimental animal, e.g. a mouse or a rat, a domestic animal, or a non-human primate. The experimental animal can be an animal model for a neurodegenerative disorder, e.g. a transgenic mouse and/or a knock-out mouse with an AD-type neuropathology.

In a further aspect, the invention features a modulator of an activity, or a level, or both said activity and said level of at least one substance which is selected from the group consisting of (i) a gene coding for a nuclear restricted protein, and/or (ii) a transcription product of a gene coding for a nuclear restricted protein, and/or (iii) a translation product of a gene coding for a nuclear restricted protein, and/or (iv) a fragment or derivative of (i) to (iii).

In an additional aspect, the invention features a pharmaceutical composition comprising said modulator and preferably a pharmaceutical carrier. Said carrier refers to a diluent, adjuvant, excipient, or vehicle with which the modulator is administered.

In a further aspect, the invention features a modulator of an activity, or a level, or both said activity and said level of at least one substance which is selected from the group consisting of (i) a gene coding for a nuclear restricted protein, and/or (ii) a transcription product of a gene coding for a nuclear restricted protein, and/or (iii) a translation product of a gene coding for a nuclear restricted protein, and/or (iv) a fragment or derivative of (i) to (iii) for use in a pharmaceutical composition.

In another aspect, the invention provides for the use of a modulator of an activity, or a level, or both said activity and said level of at least one substance which is selected from the group consisting of (i) a gene coding for a nuclear restricted protein, and/or (ii) a transcription product of a gene coding for a nuclear restricted protein, and/or (iii) a translation product of a gene coding for a nuclear restricted protein, and/or (iv) a fragment or derivative of (i) to (iii) for a preparation of a medicament for treating or preventing a neurodegenerative disease, in particular AD.

In one aspect, the present invention also provides a kit comprising one or more containers filled with a therapeutically or prophylactically effective amount of said pharmaceutical composition.

In a further aspect, the invention features a recombinant, non-human animal comprising a non-native gene sequence coding for a nuclear restricted protein, or a fragment thereof, or a derivative thereof. The generation of said recombinant, non-human animals comprises (i) providing a gene targeting construct containing said gene sequence and a selectable marker sequence, and (ii) introducing said targeting construct into a stem cell of a non-human animal, and (iii) introducing said non-human animal stem cell into a non-human embryo, and (iv) transplanting said embryo into a pseudopregnant non-human animal, and (v) allowing said embryo to develop to term, and (vi) identifying a genetically altered non-human animal whose genome comprises a modification of said gene sequence in both alleles, and (vii) breeding the genetically altered non-human animal of step (vi) to obtain a genetically altered non-human animal whose genome comprises a modification of said endogenous gene, wherein said gene is mis-expressed, or under-expressed, or over-expressed, and wherein said disruption or alteration results in said non-human animal exhibiting a predisposition to developing symptoms of neuropathology similar to a neurodegenerative disease, in particular AD. Strategies and techniques for the generation and construction of such an animal are known to those of ordinary skill in the art (see e.g. Capecchi, Science, 1989, 244:1288-1292 and Hogan et al., 1994, Manipulating the Mouse Embryo: A Laboratory Manual, Cold Spring Harbor Laboratory Press, Cold Spring Harbor, N.Y.). In preferred embodiments, said recombinant, non-human animal comprises a non-native gene sequence coding for the nuclear restricted protein/brain, NRP/B, or a fragment thereof.

In another aspect, the invention features an assay for screening for a modulator of neurodegenerative diseases, in particular AD, or related diseases and disorders of one or more substances selected from the group consisting of (i) a gene coding for a nuclear restricted protein, and/or (ii) a transcription product of a gene coding for a nuclear restricted protein, and/or (iii) a translation product of a gene coding for a nuclear restricted protein, and/or (iv) a fragment or derivative of (i) to (iii). This screening method comprises (a) contacting a cell with a test compound, and (b) measuring the activity, or the level, or both the activity and the level of one or more substances recited in (i) to (iv), and (c) measuring the activity, or the level, or both the activity and the level of said substances in a control cell not contacted with said test compound, and (d) comparing the levels of the substance in the cells of step (b) and (c), wherein an alteration in the activity and/or level of said substances in the contacted cells indicates that the test compound is a modulator of said diseases and disorders.

In one further aspect, the invention features a screening assay for a modulator of neurodegenerative diseases, in particular AD, or related diseases and disorders of one or more substances selected from the group consisting of (i) a gene coding for a nuclear restricted protein, and/or (ii) a transcription product of a gene coding for a nuclear restricted protein, and/or (iii) a translation product of a gene coding for a nuclear restricted protein, and/or (iv) a fragment or derivative of (i) to (iii), comprising (a) administering a test compound to a test animal which is predisposed to developing or has already developed a neurodegenerative disease or related diseases or disorders, and (b) measuring the activity and/or level of one or more substances recited in (i) to (iv), and (c) measuring the activity and/or level of said substances in a matched control animal which is equally predisposed to developing or has already developed said diseases and to which animal no such test compound has been administered, and (d) comparing the activity and/or level of the substance in the animals of step (b) and (c), wherein an alteration in the activity and/or level of substances in the test animal indicates that the test compound is a modulator of said diseases and disorders.

In a preferred embodiment, said test animal and/or said control animal is a recombinant, non-human animal which expresses a nuclear restricted protein, or a fragment thereof, or a derivative thereof, under the control of a transcriptional regulatory element which is not the native nuclear restricted protein gene transcriptional control regulatory element.

In another embodiment, the present invention provides a method for producing a medicament comprising the steps of (i) identifying a modulator of neurodegenerative diseases by a method of the aforementioned screening assays and (ii) admixing the modulator with a pharmaceutical carrier. However, said modulator may also be identifiable by other types of screening assays.

In another aspect, the present invention provides for an assay for testing a compound, preferably for screening a plurality of compounds, for inhibition of binding between a ligand and a nuclear restricted protein, or a fragment or derivative thereof. Said screening assay comprises the steps of (i) adding a liquid suspension of said nuclear restricted protein, or a fragment or derivative thereof, to a plurality of containers, and (ii) adding a compound or a plurality of compounds to be screened for said inhibition to said plurality of containers, and (iii) adding fluorescently labelled ligand to said containers, and (iv) incubating said nuclear restricted protein, or said fragment or derivative thereof, and said compound or plurality of compounds, and said fluorescently labelled ligand, and (v) measuring the amounts of fluorescence associated with said nuclear restricted protein, or with said fragment or derivative thereof, and (vi) determining the degree of inhibition by one or more of said compounds of binding of said ligand to said nuclear restricted protein, or said fragment or derivative thereof. Instead of utilizing a fluorescently labelled ligand, it might in some aspects be preferred to use any other detectable label known to the person skilled in the art, e.g. radioactive labels, and detect it accordingly. Said method may be useful for the identification of novel compounds as well as for evaluating compounds which have been improved or otherwise optimized in their ability to inhibit the binding of a ligand to a gene product of a gene coding for a nuclear restricted protein, or a fragment or derivative thereof. In one further embodiment, the present invention provides a method for producing a medicament comprising the steps of (i) identifying a compound as an inhibitor of binding between a ligand and and a gene product of a gene coding for a nuclear restricted protein by the aforementioned inhibitory biding assay and (ii) admixing the compound with a pharmaceutical carrier. However, said compound may also be identifiable by other types of screening assays.

In another aspect, the invention features an assay for testing a compound, preferably for screening a plurality of compounds, to determine the degree of binding of said compounds to a nuclear restricted protein, or to a fragment or derivative thereof. Said screening assay comprises (i) adding a liquid suspension of said nuclear restricted protein, or a fragment or derivative thereof, to a plurality of containers, and (ii) adding a fluorescently labelled compound or a plurality of fluorescently labelled compounds to be screened for said binding to said plurality of containers, and (iii) incubating said nuclear restricted protein, or said fragment or derivative thereof, and said fluorescently labelled compounds, and (iv) measuring the amounts of fluorescence associated with said nuclear restricted protein, or with said fragment or derivative thereof, and (v) determining the degree of binding by one or more of said compounds to said nuclear restricted protein, or said fragment or derivative thereof. In this type of assay it might be preferred to use a fluorescent label. However, any other type of detectable label might also be employed. Said method may be useful for the identification of novel compounds as well as for evaluating compounds which have been improved or otherwise optimized in their ability to bind to a nuclear restricted protein. In one further embodiment, the present invention provides a method for producing a medicament comprising the steps of (i) identifying a compound as an inhibitor of binding between a ligand and and a gene product of a gene coding for a nuclear restricted protein by the aforementioned inhibitory biding assay and (ii) admixing the compound with a pharmaceutical carrier. However, said compound may also be identifiable by other types of screening assays. In one further embodiment, the present invention provides a method for producing a medicament comprising the steps of (i) identifying a compound as a binder to a gene product of gene coding for a nuclear restricted protein by the aforementioned binding assay and (ii) admixing the compound with a pharmaceutical carrier. However, said compound may also be identifiable by other types of screening assays.

It is one further embodiment of the present invention to provide a medicament obtainable by any of the methods according to the herein claimed screening assays. In another embodiment, the instant invention provides for a medicament obtained by any of the methods according to the herein claimed screening assays.

In all types of assays disclosed herein it is preferred to study and conduct screening assays with the nuclear restricted protein/brain, NRP/B.

The present invention features an antibody which is specifically immunoreactive with an immunogen, wherein said immunogen is a translation product of a gene coding for a nuclear restricted protein, in particular the nuclear restricted protein/brain, or a fragment thereof. The immunogen may comprise immunogenic or antigenic epitopes or portions of a translation product of said gene, wherein said immunogenic or antigenic portion of a translation product is a polypeptide-, and wherein said polypeptide elicits an antibody response in an animal, and wherein said polypeptide is immunospecifically bound by said antibody. Methods for generating antibodies are well known in the art (see Harlow et al., Antibodies, A Laboratory Manual, Cold Spring Harbor Laboratory Press, Cold Spring Harbor, N.Y.). The term “antibody”, as employed in the present invention, encompasses all forms of antibodies known in the art, such as polyclonal, monoclonal, chimeric, recombinatorial, anti-idiotypic, humanized, or single chain antibodies, as well as fragments thereof. Antibodies of the present invention are useful, for instance, in a variety of diagnostic and therapeutic methods involving detecting translation products of a gene coding for a nuclear restricted protein, in particular the nuclear restricted protein/brain, NRP/B.

In a preferred embodiment of the present invention, said antibodies can be used for detecting the pathological state of a cell in a sample from a subject, comprising immunocytochemical staining of said cell with said antibody, wherein an altered degree of staining, or an altered staining pattern in said cell compared to a cell representing a known health status indicates a pathological state of said cell. Preferably, the pathological state relates to a neurodegenerative disease, in particular to AD. Immunocytochemical staining of a cell can be carried out by a number of different experimental methods well known in the art. It might be preferred, however, to apply an automated method for the detection of antibody binding, wherein the determination of the degree of staining of a cell, or the determination of the cellular or subcellular staining pattern of a cell, or the topological distribution of an antigen on the cell surface or among organelles and other subcellular structures within the cell, are carried out according to the method described in U.S. Pat. No. 6,150,173).

Other features and advantages of the invention will be apparent from the following description of figures and examples which are illustrative only and not intended to limit the remainder of the disclosure in any way.

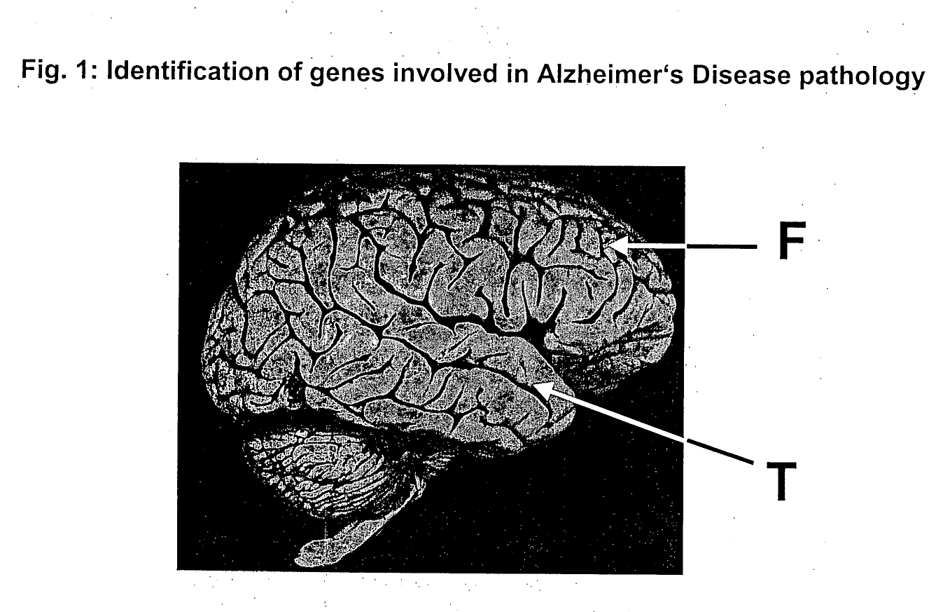

FIG. 1 depicts the brain regions with selective vulnerability to neuronal loss and degeneration in AD. Primarily, neurons within the inferior temporal lobe, the entorhinal cortex, the hippocampus, and the amygdala are subject to degenerative processes in AD (Terry et al., Annals of Neurology 1981, 10:184-192). These brain regions are mostly involved in the processing of learning and memory functions. In contrast, neurons within the frontal cortex, the occipital cortex, and the cerebellum remain largely intact and preserved from neurodegenerative processes in AD. Brain tissues from the frontal cortex (F) and the temporal cortex (T) of AD patients and healthy, age-matched control individuals was used for the herein disclosed examples. For illustrative purposes, the image of a healthy brain was taken from a publication by Strange (Brain Biochemistry and Brain Disorders, Oxford University Press, Oxford, 1992, p.4).

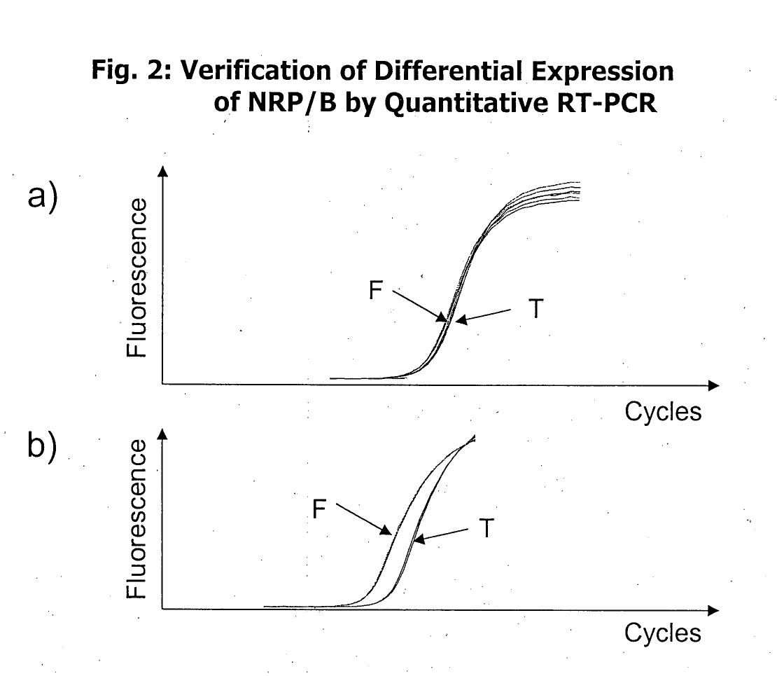

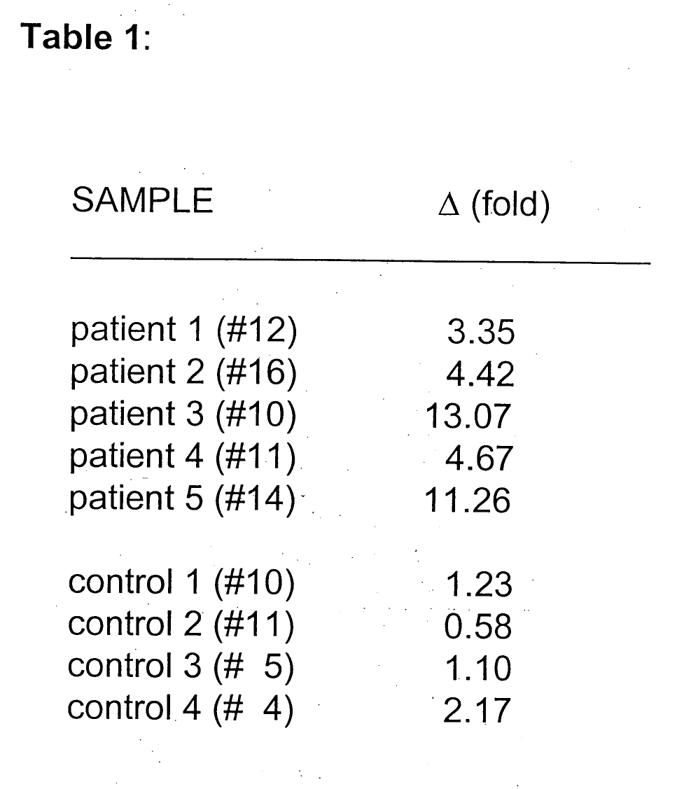

FIG. 2 illustrates the verification of the differential expression of NRP/B by quantitative RT-PCR analysis. Quantification of RT-PCR products from RNA samples collected from the frontal cortex (F) and temporal cortex (T) of healthy, age-matched control individuals (FIG. 2a) and AD patients (FIG. 2b) was performed by the LightCycler rapid thermal cycling technique. The data were normalized to cyclophilin B which showed no significant difference in its gene expression level. The figure depicts the kinetics of amplification by plotting the cycle number against the amount of amplified material as measured by its fluorescence. The amplification kinetics of NRP/B cDNA from both the frontal and temporal cortices of a normal control individual during the exponential phase of the reaction overlap (FIG. 2a), whereas in AD there is a significant shift of the curve for the sample derived from frontal cortex (FIG. 2b), indicating an up-regulation of NRP/B mRNA in the frontal cortex relative to temporal cortex.

Table 1 lists the gene expression levels in the frontal cortex relative to the temporal cortex for the gene coding for the nuclear restricted protein/brain, NRP/B, in five AD patients (3.35 to 13.07 fold) and four healthy, age-matched control individuals (0.58 to 2.17 fold). The values shown are reciprocal values according to the formula described herein (see below).

### EXAMPLE I

(i) Brain Tissue Dissection from Patients With AD:

Brain tissues from AD patients and age-matched control subjects were obtained from qualified institutions and brain banks. The tissue was collected within 6 hours post-mortem and immediately frozen on dry ice. Sample sections from each tissue were fixed in paraformaldehyde for histo-pathological confirmation of the diagnosis. Brain areas for differential expression analysis were identified (see FIG. 1) and stored at −80° C. until RNA extractions were performed.

(ii) Isolation of Total mRNA:

Total RNA was extracted from post-mortem brain tissue by using the RNeasy kit (Qiagen) according to the manufacturer's protocol. The quality of the prepared RNA was determined by formaldehyde agarose gel electrophoresis and Northern blotting according to standard procedures (Sambrook and Russell, Molecular Cloning: A Laboratory Manual, Cold Spring Harbor Laboratory Press, Cold Spring Harbor, N.Y., 2000). The mRNA was isolated from the total RNA preparation using the Quick-prep Micro mRNA Purification Kit (Pharmacia Biotech).

(iii) cDNA Synthesis and Identification of Differentially Expressed Genes by Suppressive Subtractive Hybridization:

This technique compares two populations of mRNA and provides clones of genes that are expressed in one population but not in the other. The applied technique was described in detail by Diatchenko et al. (Proc Natl Acad Sci USA 1996, 93:6025-30). In the present invention, mRNA populations from post-mortem brain tissues from AD patients were compared. Specifically, mRNA of the frontal cortex was subtracted from mRNA of the inferior temporal cortex. The necessary reagents were taken from the PCR-Select cDNA subtraction kit (Clontech), and all steps were performed as described in the manufacturer's protocol. Specifically, 2 μg mRNA each were used for first-strand and second-strand cDNA synthesis. After RsaI-digestion and adaptor ligation hybridization of tester and driver was performed for 8 hours (first hybridization) and 15 hours (second hybridization) at 68° C. Two PCR steps were performed to amplify differentially expressed genes (first PCR: 27 cycles of 94° C. and 30 sec, 66° C. and 30 sec, and 72° C. and 1.5 min; nested PCR: 12 cycles of 94° C. and 30 sec, 66° C. and 30 sec, and 72° C. and 1.5 min) using adaptor specific primers (included in the subtraction kit) and 50× Advantage Polymerase Mix (Clontech). Efficiencies of RsaI-digestions, adaptor ligations and subtractive hybridizations were checked as recommended in the kit. Subtracted cDNAs were inserted into the pCR® vector and transformed into E. coli INVaF′ cells (Invitrogen). To isolate individual cDNAs of the subtracted library single bacterial transformants were incubated in 100 μl LB (with 50 μg/ml ampicillin) at 37° C. for at least 4 hours. Inserts were PCR amplified (95° C. and 30 sec, 68° C. and 3 min for 30 cycles) in a volume of 20 μl containing 10 mM Tris-HCl pH 9.0, 1.5 mM MgCl₂, 50 mM KCl, 200 μM dNTP, 0.5 μM adaptor specific primers (included in the subtraction kit), 1.5 Units Taq polymerase (Pharmacia Biotech), and 1 μl of bacterial culture. An aliquot of the mixture (1.5 μl) containing 3 μl PCR amplified inserts and 2 μl 0.3 N NaOH/15% Ficoll were spotted onto a positively charged nylon membrane (Roche). In this way, hundreds of spots were arrayed on duplicate filters for subsequent hybridization. The differential screening step consisted of hybridizations of the subtracted library with itself to minimize background (Wang and Brown, Proc Natl Acad Sci USA 1991, 88:11505-9). The probes were made of the nested PCR product of the subtraction following the instructions of the Clontech subtraction kit. Labeling with digoxigenin was performed with the DIG DNA Labeling Kit (Roche). Hybridizations were carried out overnight in DIG Easy HYB (Roche) at 43° C. The filters were washed twice in 2×SSC/0.5% SDS at 68° C. for 15 min and twice in 0.1×SSC/0.5% SDS at 68° C. for 15 min, and subjected to detection using anti-DIG-AP conjugates and CDPStar™ as chemiluminescent substrate according to the instructions of the DIG DNA Detection Kit (Roche). Blots were exposed to Kodak Biomax MR chemiluminescent film at room temperature for several minutes. The nucleotide sequences of clones of interest were obtained using methods well known in the art. For nucleotide sequence analyses and homology searches, computer algorithms of the University of Wisconsin Genetics Computer Group (GCG) together with publicly available nucleotide and peptide sequence information (GenBank and EMBL databases) were employed.

(iv) Confirmation of Differential Expression by Quantitative RT-PCR:

Positive confirmation of differential expression of the NRP/B gene was performed using the LightCycler technology (Roche). This technique features rapid thermal cyling for the polymerase chain reaction as well as real-time measurement of fluorescent signals during amplification and therefore allows for highly accurate quantification of RT-PCR products by using a kinetic, rather than an endpoint approach. The ratio of NRP/B cDNA from the temporal cortex and frontal cortex was determined (relative quantification). In a first step a standard curve was generated to determine the efficiency of the PCR with specific primers for NRP/B (5′ATAGGTGCTTCCCCTGAGGTG-3′ and 5′-GCAATGTGAGAAACATGGACGA-3′). PCR amplification (95° C. and 1 sec, 56° C. and 5 sec, and 72° C. and 5 sec) was performed in a volume of 20 μl containing Lightcycler-DNA Master SYBR Green mix (containing Taq DNA polymerase, reaction buffer, dNTP mix with dUTP instead of dTTP, SYBR Green I dye, and 1 mM MgCl₂, Roche), additionally containing 3 mM MgCl₂, 0.5 μM primers, 0.16 μl TaqStart® antibody (Clontech), and 1 μl of a cDNA dilution series (40, 20, 10, 5, and 1 ng human total brain cDNA, Clontech). Melting curve analysis revealed a single peak at approximately 83° C. with no visible primer dimers. Agarose gel analysis of the PCR product showed one single band of the expected size (78 bp). The same protocol was applied to determine the PCR efficiency of the reference gene, cyclophilin B, using the specific primers 5′-ACTGAAGCACTACGGGCCTG-3′ and 5′-AGCCGTTGGTGTCTTTGCC-3′ except for MgCl₂ (an additional 1 mM was added instead of 3 mM). Cyclophilin-B was chosen for normalization because it was found to be the least regulated gene among all analyzed housekeeping genes. Melting curve analysis revealed a single peak at approximately 87° C. with no visible primer dimers. Agarose gel analysis of the PCR product showed one single band of the expected size (62 bp). The logarithm of the cDNA concentration was plotted against the threshold cycle number Cₜ for both NRP/B and cyclophilin B. The slopes and the intercepts of the standard curves (i.e. linear regressions) were calculated for both genes. In a second step, cDNA from temporal cortex and frontal cortex was analyzed in parallel with cyclophilin B for normalization. The Cₜ values were measured and converted to ng total brain cDNA using the corresponding standard curves:

10\hat{ }((Cₜ value−intercept)/slope)[ng total brain cDNA]

The values of temporal and frontal cortex NRP/B cDNAs were normalized to cyclophilin B and the ratio was calculated using the following formula:

$$Ratio = \frac{NRP / B temporal [ ng ] / cyclophilin B temporal [ ng ]}{NRP / B frontal [ ng ] / cyclophilin B frontal [ ng ]}$$

The results of one such quantitative RT-PCR analysis for the NRP/B gene are shown in FIG. 2.

1. A method of diagnosing or prognosticating a neurodegenerative disease in a subject, or determining whether a subject is at increased risk of developing said disease, comprising determining a level and/or an activity of
(i) a transcription product of a gene coding for NRP/B, and/or
(ii) a translation product of a gene coding for NRP/B, and/or
(iii) a fragment or derivative of said transcription or translation product,
in a sample from said subject and comparing said level and/or said activity to a reference value representing a known disease or health status, thereby diagnosing or prognosticating said neurodegenerative disease in said subject, or determining whether said subject is at increased risk of developing said neurodegenerative disease.

2. A method of monitoring the progression of a neurodegenerative disease in a subject, comprising determining a level and/or an activity of
(i) a transcription product of a gene coding for NRP/B, and/or
(ii) a translation product of a gene coding for NRP/B, and/or
(iii) a fragment or derivative of said transcription or translation product,
in a sample from said subject and comparing said level and/or said activity to a reference value representing a known disease or health status, thereby monitoring the progression of said neurodegenerative disease in said subject.

3. A method of evaluating a treatment for a neurodegenerative disease, comprising determining a level and/or an activity of
(i) a transcription product of a gene coding for NRP/B, and/or
(ii) a translation product of a gene coding for NRP/B, and/or
(iii) a fragment or derivative of said transcription or translation product,
in a sample from a subject being treated for said disease and comparing said level and/or said activity to a reference value representing a known disease or health status, thereby evaluating said treatment for said neurodegenerative disease.

4. The method according to any of claims 1 to 3 wherein said neurodegenerative disease is Alzheimer's disease.

5. The method according to any of claims 1 to 4 wherein said sample comprises a cell, or a tissue, or an organ, or a body fluid, in particular cerebrospinal fluid or blood.

6. The method according to any of claims 1 to 5 wherein said reference value is that of a level and/or an activity of
(i) a transcription product of a gene coding for NRP/B, and/or
(ii) a translation product of a gene coding for NRP/B, and/or
(iii) a fragment or derivative of said transcription or translation product,
in a sample from a subject not suffering from said neurodegenerative disease.

7. The method according to any of claims 1 to 6 wherein an increase or decrease in a transcription product of the gene coding for NRP/B and/or a translation product of a gene coding for NRP/B in a cell, or tissue, or body fluid, in particular cerebrospinal fluid, from said subject relative to a reference value representing a known health status indicates a diagnosis, or prognosis, or increased risk of Alzheimer's disease in said subject.

8. The method according to any of claims 1 to 7, further comprising comparing a level and/or an activity of
(i) a transcription product of a gene coding for NRP/B, and/or
(ii) a translation product of a gene coding for NRP/B, and/or
(iii) a fragment or derivative of said transcription or translation product
in a series of samples taken from said subject over a period of time.

9. The method according to claim 8 wherein said subject receives a treatment prior to one or more of said sample gatherings.

10. The method according to claim 9 wherein said level and/or activity is determined before and after said treatment of said subject.

11. A kit for diagnosing or prognosticating a neurodegenerative disease, in particular Alzheimer's disease, in a subject, or determining the propensity or predisposition of a subject to develop such a disease, said kit comprising:
(a) at least one reagent which is selected from the group consisting of (i) reagents that selectively detect a transcription product of a gene coding for NRP/B and (ii) reagents that selectively detect a translation product of a gene coding for NRP/B and
(b) an instruction for diagnosing, or prognosticating a neurodegenerative disease, in particular Alzheimer's disease, or determining the propensity or predisposition of a subject to develop such a disease by (i) detecting a level, or an activity, or both said level and said activity, of said transcription product and/or said translation product of a gene coding for NRP/B, in a sample from said subject; and (ii) diagnosing or prognosticating a neurodegenerative disease, in particular Alzheimer's disease, or determining the propensity or predisposition of said subject to develop such a disease, wherein a varied level, or activity, or both said level and said activity, of said transcription product and/or said translation product compared to a reference value representing a known health status; or a level, or activity, or both said level and said activity, of said transcription product and/or said translation product similar or equal to a reference value representing a known disease status indicates a diagnosis or prognosis of a neurodegenerative disease, in particular Alzheimer's disease, or an increased propensity or predisposition of developing such a disease.

12. A method of treating or preventing a neurodegenerative disease, in particular Alzheimer's disease, in a subject comprising administering to said subject in a therapeutically or prophylactically effective amount an agent or agents which directly or indirectly affect an activity and/or a level of (i) a gene coding for NRP/B, and/or (ii) a transcription product of a gene coding for NRP/B, and/or (iii) a translation product of a gene coding for NRP/B, and/or (iv) a fragment or derivative of (i) to (iii).

13. A modulator of an activity and/or of a level of at least one substance which is selected from the group consisting of (i) a gene coding for NRP/B, and/or (ii) a transcription product of a gene coding for NRP/B, and/or (iii) a translation product of a gene coding for NRP/B, and/or (iv) a fragment or derivative of (i) to (iii).

14. A pharmaceutical composition comprising a modulator according to claim 13.

15. A modulator of an activity and/or of a level of at least one substance which is selected from the group consisting of (i) a gene coding for NRP/B, and/or (ii) a transcription product of a gene coding for NRP/B, and/or (iii) a translation product of a gene coding for NRP/B, and/or (iv) a fragment or derivative of (i) to (iii) for use in a pharmaceutical composition.

16. Use of a modulator of an activity and/or of a level of at least one substance which is selected from the group consisting of (i) a gene coding for NRP/B, and/or (ii) a transcription product of a gene coding for NRP/B, and/or (iii) a translation product of a gene coding for NRP/B, and/or (iv) a fragment or derivative of (i) to (iii) for a preparation of a medicament for treating or preventing a neurodegenerative disease, in particular Alzheimer's disease.

17. A kit, comprising in one or more containers, a therapeutically or prophylactically effective amount of the pharmaceutical composition of claim 14.

18. A recombinant, non-human animal comprising a non-native gene sequence coding for NRP/B, or a fragment thereof, or a derivative thereof, said animal being obtainable by:
(i) providing a gene targeting construct comprising said gene sequence and a selectable marker sequence, and
(ii) introducing said targeting construct into a stem cell of a non-human animal, and
(iii) introducing said non-human animal stem cell into a non-human embryo, and
(iv) transplanting said embryo into a pseudopregnant non-human animal, and
(v) allowing said embryo to develop to term, and
(vi) identifying a genetically altered non-human animal whose genome comprises a modification of said gene sequence in both alleles, and
(vii) breeding the genetically altered non-human animal of step (vi) to obtain a genetically altered non-human animal whose genome comprises a modification of said endogenous gene, wherein said disruption results in said non-human animal exhibiting a predisposition to developing a neurodegenerative disease or related disease or disorders.

19. An assay for screening for a modulator of neurodegenerative diseases, in particular Alzheimer's disease, or related diseases or disorders of one or more substances selected from the group consisting of
(i) a gene coding for NRP/B, and/or
(ii) a transcription product of a gene coding for NRP/B, and/or
(iii) a translation product of a gene coding for NRP/B, and/or
(iv) a fragment or derivative of (i) to (iii), said method comprising:
(a) contacting a cell with a test compound;
(b) measuring the activity and/or level of one or more substances recited in (i) to (iv);
(c) measuring the activity and/or level of one or more substances recited in (i) to (iv) in a control cell not contacted with said test compound; and
(d) comparing the levels and/or activities of the substance in the cells of step (b) and (c), wherein an alteration in the activity and/or level of substances in the contacted cells indicates that the test compound is a modulator of said diseases or disorders.

20. A method of screening for a modulator of neurodegenerative diseases, in particular Alzheimer's disease, or related diseases or disorders of one or more substances selected from the group consisting of
(i) a gene coding for NRP/B, and/or
(ii) a transcription product of a gene coding for NRP/B, and/or
(iii) a translation product of a gene coding for NRP/B, and/or
(iv) a fragment or derivative of (i) to (iii), said method comprising:
(a) administering a test compound to a test animal which is predisposed to developing or has already developed a neurodegenerative disease or related diseases or disorders in respect of the substances recited in (i) to (iv);
(b) measuring the activity and/or level of one or more substances recited in (i) to (iv);
(c) measuring the activity and/or level of one or more substances recited in (i) or (iv) in a matched control animal which is predisposed to developing or has already developed a neurodegenerative disease or related diseases or disorders in respect to the substances recited in (i) to (iv) and to which animal no such test compound has been administered;
(d) comparing the activity and/or level of the substance in the animals of step (b) and (c), wherein an alteration in the activity and/or level of substances in the test animal indicates that the test compound is a modulator of said diseases or disorders.

21. The method according to claim 20 wherein said test animal and/or said control animal is a recombinant animal which expresses NRP/B, or a fragment thereof, or a derivative thereof, under the control of a transcriptional control element which is not the native NRP/B gene transcriptional control element.

22. An assay for testing a compound, preferably for screening a plurality of compounds for inhibition of binding between a ligand and NRP/B, or a fragment or derivative thereof, said assay comprising the steps of:
(i) adding a liquid suspension of NRP/B, or a fragment or derivative thereof, to a plurality of containers;
(ii) adding a plurality of compounds to be screened for said inhibition to said plurality of containers;
(iii) adding fluorescently labelled ligand to said containers;
(iv) incubating said NRP/B, or said fragment or derivative thereof, and said compounds, and said fluorescently labelled ligand;
(v) measuring amounts of fluorescence associated with said NRP/B, or with said fragment or derivative thereof; and
(vi) determining the degree of inhibition by one or more of said compounds of binding of said ligand to said NRP/B, or said fragment or derivative thereof.

23. An assay for testing a compound, preferably for screening a plurality of compounds to determine the degree of binding of said compounds to NRP/B, or to a fragment or derivative thereof, said assay comprising the steps of:
(i) adding a liquid suspension of NRP/B, or a fragment or derivative thereof, to a plurality of containers;
(ii) adding a fluorescently labelled compound or a plurality of fluorescently labelled compounds to be screened for said binding to said plurality of containers;
(iii) incubating said NRP/B, or said fragment or derivative thereof, and said fluorescently labelled compounds;
(iv) measuring amounts of fluorescence associated with said NRP/B, or with said fragment or derivative thereof; and
(v) determining the degree of binding by one or more of said compounds to said NRP/B, or said fragment or derivative thereof.

24. Use of an antibody specifically immunoreactive with an immunogen, wherein said immunogen is a translation product of a gene coding for NRP/B, or a fragment thereof, for detecting the pathological state of a cell in a sample from a subject, comprising immunocytochemical staining of said cell with said antibody, wherein an altered degree of staining, or an altered staining pattern in said cell compared to a cell representing a known health status indicates a pathological state of said cell, and wherein said pathological state relates to a neurodegenerative disease, in particular Alzheimer's disease.
